(19) United States
(12) Patent Application Publication
(10) Pub. No.: US 2025/0295931 A1
(43) Pub. Date: Sep. 25, 2025

(54) CANCER TREATMENT SYSTEM

(71) Applicants: Ricoh Company, Ltd., Tokyo (JP); Public University Corporation Yokohama City University, Kanagawa (JP)

(72) Inventors: Takashi YAMAGUCHI, Kanagawa (JP); Kazuhito KISHI, Kanagawa (JP); Motokazu HASEGAWA, Kanagawa (JP); Masanari UMEMURA, Kanagawa (JP); Yoshihiro ISHIKAWA, Kanagawa (JP); Akane NAGASAKO, Kanagawa (JP)

(73) Assignees: Ricoh Company, Ltd., Tokyo (JP); Public University Corporation Yokohama City University, Kanagawa (JP)

(21) Appl. No.: 18/860,238

(22) PCT Filed: Apr. 25, 2023
(86) PCT No.: PCT/JP2023/016300

§ 371 Date: Oct. 25, 2024

(30) Foreign Application Priority Data
Apr. 28, 2022 (JP) 2022-075118
Apr. 28, 2022 (JP) 2022-075130

(51) Int. Cl. A61N 2/00; A61N 2/02

(52) U.S. Cl. CPC A61N 2/004; A61N 2/02

(57) ABSTRACT

To provide a cancer treatment device that monitors a magnetic field applied to an affected area or monitors a temperature of the affected area or around the affected area. [Means of Achieving the Object] A cancer treatment system includes a magnetic field generator configured to generate an alternating current magnetic field to be applied to a cancer affected area, a magnetic field measurer configured to measure a magnitude of the magnetic field generated by the magnetic field generator, wherein the cancer affected area is inserted into the magnetic field generated by the magnetic field generator to treat cancers.

### TECHNICAL FIELD

The present disclosure relates to a cancer treatment system.

### BACKGROUND ART

Various types of cancer treatment devices are known as treatment devices used for treatment of cancer.

Patent Document 1 and Patent Document 2 disclose a cancer treatment device for treating cancer by an alternating current magnetic field.

### SUMMARY OF INVENTION

### Technical Problem

In the cancer treatment devices disclosed in Patent Document 1 and Patent Document 2, when a magnetic field is applied to an affected area by an alternating current magnetic field, the magnitude of the magnetic field itself applied to the affected area is effective in suppressing proliferation of cancer cells. When the cancer treatment device is used, the temperature around the affected area to which the alternating current magnetic field is applied may increase, and therefore monitoring the temperature is necessary. When the cancer treatment device is used, it is also necessary to monitor how much and for how many hours the magnetic field is applied to the affected area in a cumulative manner, in order to suppress proliferation of cancer cells in the affected part.

The present disclosure provides a cancer treatment device that monitors a magnetic field applied to an affected area and/or monitors an increase in temperature of the affected area.

### Solution to Problem

According to embodiments of the present invention, a cancer treatment system includes a magnetic field generator configured to generate an alternating current magnetic field to be applied to a cancer affected area, a magnetic field measurer configured to measure a magnitude of the magnetic field generated by the magnetic field generator, wherein the cancer affected area is inserted into the magnetic field generated by the magnetic field generator to treat cancers. A cancer treatment system includes a magnetic field generator configured to generate an alternating current magnetic field to be applied to a cancer affected area, a temperature measurer configured to measure a temperature of the cancer affected area or around the cancer affected area, wherein the cancer affected area is inserted into the magnetic field generated by the magnetic field generator to treat cancers without using a heat-generating medium and without depending on hyperthermic effect using a heat-generating action by the magnetic field.

### Advantageous Effects of Invention

According to the cancer treatment device of the present disclosure, the magnetic field applied to the affected area can be monitored, and/or the temperature rise of the affected area can be monitored.

### BRIEF DESCRIPTION OF DRAWINGS

FIG. 1 is a top view of a cancer treatment device in use according to the present embodiment;

FIG. 2 is a front view of the cancer treatment device in use according to the present embodiment;

FIG. 3 is a side view of the cancer treatment device in use according to the present embodiment;

FIG. 4 is a perspective view of a magnetic field generator in the cancer treatment device according to the present embodiment;

FIG. 5 is a cross-sectional perspective view of the magnetic field generator in the cancer treatment device according to the present embodiment;

FIG. 6 is a perspective view of a magnetic field measurer in the cancer treatment system according to the first embodiment;

FIG. 7 is a top view illustrating magnetic field measurement in the cancer treatment system according to the first embodiment;

FIG. 8 is a top view illustrating magnetic field measurement in the cancer treatment system according to the first embodiment;

FIG. 9 is a flowchart illustrating magnetic field measurement processing in the cancer treatment system according to the first embodiment;

FIG. 10 is a side view illustrating temperature measurement in the cancer treatment system according to the second embodiment;

FIG. 11 is a side view illustrating temperature measurement in the cancer treatment system according to the second embodiment;

FIG. 12 is a front view illustrating temperature measurement in the cancer treatment system according to the second embodiment;

FIG. 13 is a flowchart illustrating temperature measurement processing in the cancer treatment system according to the second embodiment;

FIG. 14 is a side view illustrating temperature measurement and magnetic field measurement in the cancer treatment system according to the modified embodiment; and

FIG. 15 is a side view illustrating temperature measurement and magnetic field measurement in the cancer treatment system according to the modified embodiment.

### DESCRIPTION OF EMBODIMENTS

Hereinafter, embodiments of the present invention will be described with reference to the drawings. In the present specification and the drawings, constituent elements having substantially the same functional configuration are denoted by the same reference numerals, and redundant description is omitted. In order to facilitate understanding, the scale of each part in the drawings may be different from the actual scale.

In directions such as parallel, right angle, orthogonal, horizontal, vertical, up-down, left-right, and the like, deviations are allowed to such an extent that the effects of the embodiments are not impaired. The shape of the corner portion is not limited to a right angle and may be rounded in an arcuate shape. Parallel, right angle, orthogonal, horizontal, and vertical may encompass substantially parallel, substantially right angle, substantially orthogonal, substantially horizontal, and substantially vertical, respectively.

### Cancer Treatment Device 10

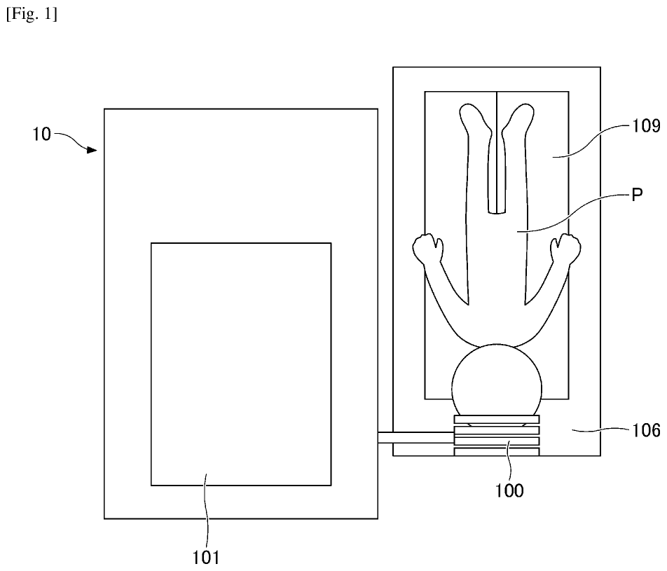

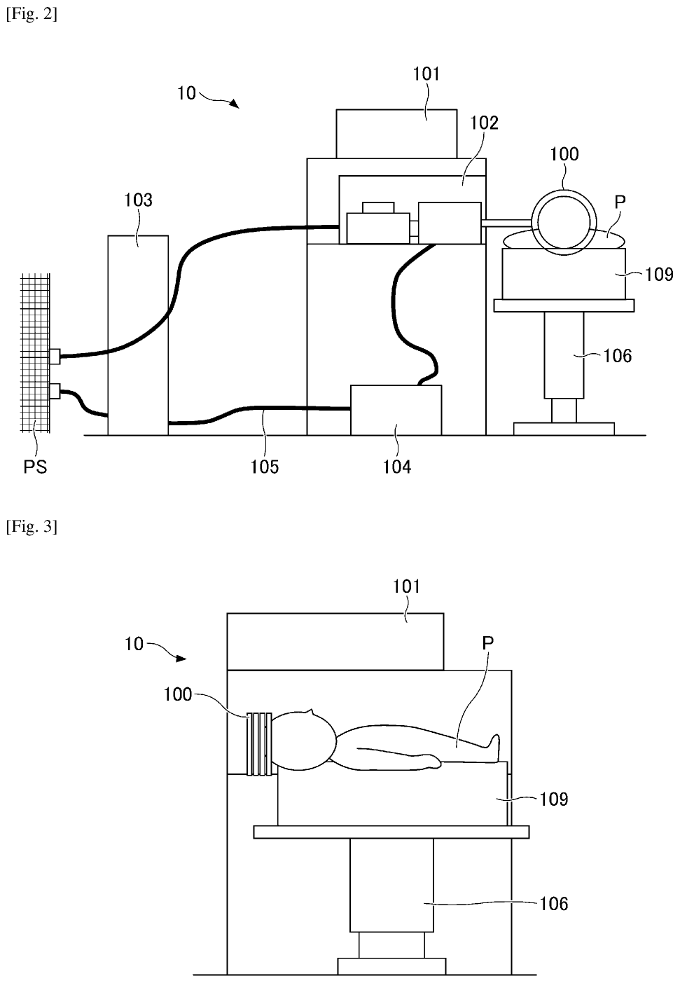

First, a cancer treatment device 10 included in a cancer treatment system according to the present embodiments will be described. FIG. 1 is a top view illustrating a cancer treatment device 10 in use according to the present embodiments. FIG. 2 is a front view illustrating cancer treatment device 10 in use according to the present embodiments. FIG. 3 is a side view illustrating the cancer treatment device 10 in use according to the present embodiments.

The cancer treatment device 10 is a cancer treatment device that suppresses proliferation of cancer cells by applying an alternating current magnetic field to a cancer affected tissue. The cancer treatment device 10 is a magnetic treatment device that applies an alternating current magnetic field generated by a coil 100a equipped in a magnetic field generator 100 to a cancer affected tissue for treatment. The cancer treatment device 10 is a cancer treatment device that treats a cancer affected area by inserting the cancer affected area into the alternating current magnetic field generated by the magnetic field generator 100.

The cancer treatment device 10 does not use a heat-generating medium. Further, the cancer treatment device 10 is not a device that treats cancers by hyperthermic effect using a heat-generating action by a magnetic field, but rather is a cancer treatment device that treats cancers by directly applying an alternating current magnetic field to a cancer affected area by the magnetic field generator 100. The cancer treatment device 10 is, for example, a cancer treatment device different from a cancer treatment device that treats cancers by hyperthermic effect using a heating effect of a magnetic field using a heat-generating medium. In other words, the cancer treatment device 10 is a cancer treatment device that treats a cancer affected area by inserting the cancer affected area into an alternating current magnetic field generated by the magnetic field generator 100 without using a heating-generating medium and without depending on hyperthermic effect using a heat-generating action by the magnetic field.

The cancer treatment device 10 includes a magnetic field generator 100, a power supply 101, a matching unit 102, and a cooler 103. The cancer treatment device 10 also includes a treatment table 106 on which a patient P is laid.

The magnetic field generator 100 generates an alternating current magnetic field to be applied to the affected area of the patient P. The magnetic field generator 100 receives current supplied from the power supply 101. The power supply 101 stably supplies a current to the magnetic field generator 100 at a target resonance frequency, for example, 230 kilohertz (kHz), by the matching unit 102. The magnetic field generator 100 converts the current supplied from the power supply 101 into a magnetic field by electromagnetic induction. The magnetic field generator 100 is formed of, for example, a coil.

The power supply 101 supplies a current for generating a magnetic field to the magnetic field generator 100. The power supply 101 is connected to a power supply PS which is, for example, a commercial power supply. The power supply 101 generates an alternating current having a frequency of, for example, 100 kHz to 300 kHz using the power supplied from the power supply PS. The power supply 101 supplies the generated alternating current to the magnetic field generator 100 via the matching unit 102. The power supply 101 adjusts the current so as to generate an alternating current magnetic field having a desired magnetic flux density in the magnetic field generator 100

The matching unit 102 performs impedance matching between the power supply 101 and the magnetic field generator 100. The matching unit 102 includes a known resonance circuit such as a series circuit or a parallel circuit including an inductor, a capacitor, and a resistor. The matching unit 102 has a control function of adjusting the frequency so that the magnetic field generator 100 can always output the maximum current value. The matching unit 102 supplies a stable current to the magnetic field generator 100.

The magnetic field generator 100 generates an alternating current magnetic field at the position where the affected part is placed, for example, the center of the coil 100a, in which a current is supplied to the magnetic field generator 100 from the power supply 101 via the matching unit 102. The magnetic flux density of the alternating current magnetic field generated by the magnetic field generator 100 is, for example, 10 to 30 millitesla (mT). The alternating current magnetic field generated by the magnetic field generator 100 may have a substantially constant intensity or may have a pattern in which a waveform accompanied by a change in a specified magnitude is repeated for a predetermined time. That is, the alternating current magnetic field generated by the magnetic field generator 100 may have a pattern in which a waveform with a change of a predetermined magnitude including a substantially constant waveform is repeated for a predetermined time.

The cooler 103 supplies a coolant to cool the magnetic field generator 100. The magnetic field generator 100 generates heat when supplied with a current from the power supply 101. The cancer treatment device 10 cools the magnetic field generator 100 with the coolant supplied by the cooler 103.

The cooler 103 supplies a coolant for cooling the magnetic field generator 100. The cooler 103 recovers the coolant that has cooled the magnetic field generator 100. The cooler 103 circulates the coolant while maintaining the liquid temperature at a constant level. The cooler 103 is, for example, a chiller. The cooler 103 is connected to a power supply PS which is, for example, a commercial power supply. The cooler 103 cools the coolant using electric power from the power supply PS. The cooler 103 supplies the coolant for cooling the magnetic field generator 100 to the magnetic field generator 100. The coolant may be, for example, water, water to which an antifreeze solution is added, or an inert liquid such as a fluorinated liquid.

The cooler 103 supplies the coolant to a manifold 104 through a hose 105. The manifold 104 supplies the coolant supplied by the cooler 103 to the magnetic field generator 100. The manifold 104 recovers the supplied coolant from the magnetic field generator 100.

In the cancer treatment device 10, the coolant supplied from the cooler 103 may be branched by the manifold 104 and supplied to, for example, the power supply 101 and the matching unit 102. That is, the cancer treatment device 10 may cool at least one of the power supply 101 and the matching unit 102 using the coolant supplied from the cooler 103.

The cancer treatment device 10 includes a non-conductive buffer material 109 on the treatment table 106. When the treatment table 106 contains metal, an eddy current may be generated by interlinkage of the alternating current magnetic field generated by the magnetic field generator 100, and the treatment table 106 may generate heat. In order to prevent heat generation of the treatment table 106, the cancer treatment device 10 includes the non-conductive buffer material 109 so as to separate the treatment table 106 from the magnetic field generator 100 by a sufficient distance. The patient P lies in a supine position on the non-conductive buffer material 109. For example, an affected area of the patient P is assumed as a brain tumor such as glioblastoma on the head. The patient P inserts his/her head into the magnetic field generator 100. The magnetic field generator 100 has a shape into which the head of the patient P can be inserted. The magnetic field generator 100 applies a magnetic field to the head of the patient P.

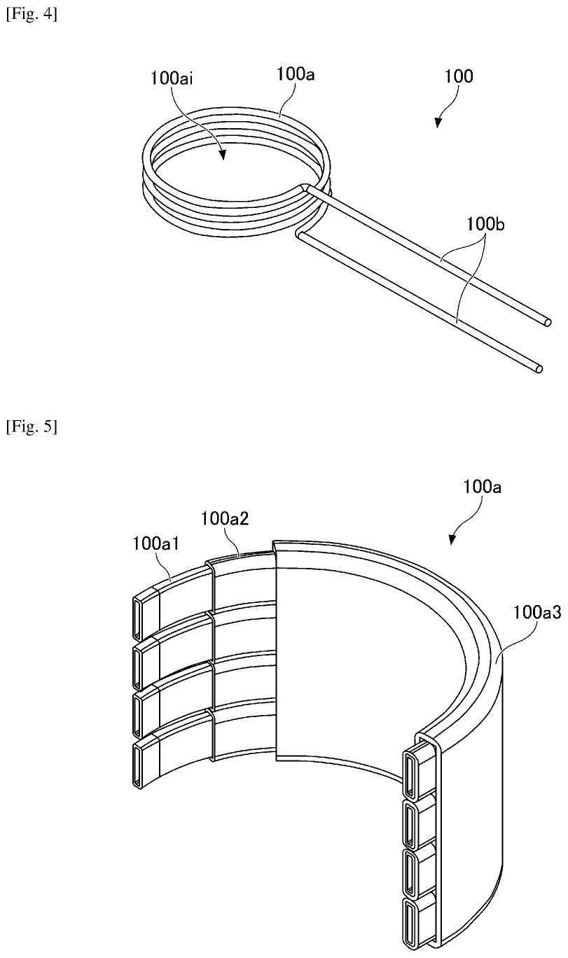

A configuration of the magnetic field generator 100 will be described. FIG. 4 is a perspective view of the magnetic field generator 100 in the cancer treatment device 10 used in the cancer treatment system according to the present embodiments.

The magnetic field generator 100 includes the coil 100a and a piping 100b. A material constituting each of the coil 100a and the piping 100b includes a material having conductivity. Each of the coil 100a and the piping 100b is constituted by a hollow tubular member. Each of the coil 100a and the piping 100b is not limited to a cylindrical shape, and may be, for example, an elliptical cylindrical shape or a rectangular cylindrical shape. FIG. 4 illustrates the coil 100a having a cylindrical shape as an example.

The coil 100a has a spiral shape. The patient P inserts the affected part into an inner side 100ai of the coil 100a. The piping 100b is connected to both ends of the coil 100a.

The temperature-controlled coolant supplied by the cooler 103 flows inside each of the coil 100a and the piping 100b. In addition, current flows from the power supply 101 to each of the coil 100a and the piping 100b via the matching unit 102. Heat generated by the current flowing through the coil 100a and the piping 100b is cooled by the coolant flowing through the coil 100a and the piping 100b. The coil 100a and the piping 100b are kept at a constant temperature by cooling with the coolant.

Next, an insulating structure included in the coil 100a will be described. When the patient P comes into contact with the coil 100a, there is a possibility that the patient P may receive electric shock. The coil 100a has an insulating structure to prevent an electric shock. FIG. 5 is a cross-sectional perspective view of the magnetic field generator 100 in the cancer treatment device 10 according to the present embodiments. FIG. 5 illustrates an example of the coil 100a having a rectangular cylindrical shape.

The coil 100a includes a metallic pipe 100a1, an insulating sheet 100a2, and a protective sheet 100a3. The metallic pipe 100a1 is made of a metallic material such as copper. The insulating sheet 100a2 is an insulating tape, for example, a polyimide tape. The protective sheet 100a3 is an insulating sheet, for example, a glass sheet. Since the coil 100a includes the insulating sheet 100a2 and the protective sheet 100a3 around the metallic pipe 100a1, the insulating sheet 100a2 and the protective sheet 100a3 protect the metallic pipe 100a1. Further, the insulating sheet 100a2 and the protective sheet 100a3 insulate the metallic pipe 100a1 from the patient P and the like.

A protective material such as cloth or rubber may be further provided on the outer side of the protective sheet 100a3 included in the coil 100a. By further providing a protective material such as cloth or rubber on the outer side of the protective sheet 100a3 included in the coil 100a, insulation from the human body can be reliably maintained.

### Cancer Treatment System 1

Next, cancer treatment system 1 according to the first embodiment will be described. The cancer treatment system 1 includes the cancer treatment device 10 and the magnetic field measurer 107. In the present disclosure, the cancer treatment system 1 is described, but the cancer treatment device 10 and the magnetic field measurer 107 may be combined to form a cancer treatment device.

Patent Document 1 discloses that the growth of cancer is suppressed when a magnetic field having a magnetic flux density of about 20 mT is applied to a cancer affected area. The cancer treatment system 1 is based on the technique disclosed in Patent Document 1 for suppressing the proliferation of cancer cells by applying a magnetic field without requiring heating of the cancer affected area. In the cancer treatment system 1, the amount of the magnetic field applied to the affected area is very important in relation to the treatment effect. Therefore, in the cancer treatment system 1, it is necessary to confirm by measurement whether or not a magnetic field having a desired magnetic flux density is being applied to the affected area.

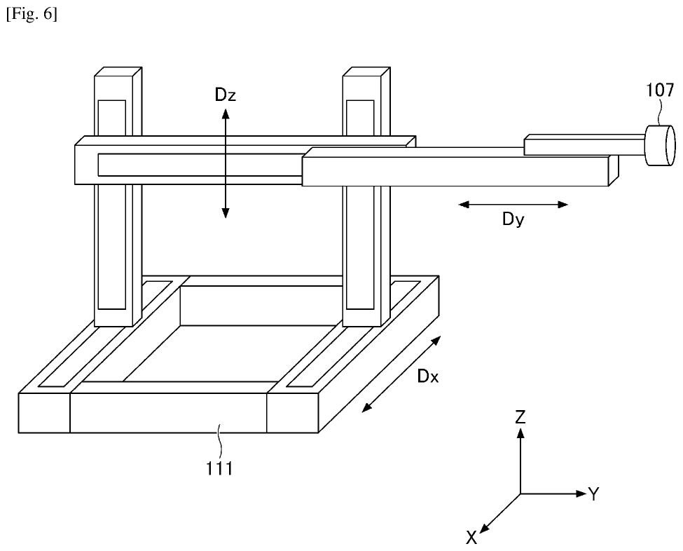

The magnetic field measurer 107 included in the cancer treatment system 1 will be described. FIG. 6 is a perspective view of the magnetic field measurer 107 in the cancer treatment system 1 according to the first embodiment.

The magnetic field measurer 107 measures the intensity of the alternating current magnetic field generated by the magnetic field generator 100. The magnetic field measurer 107 is, for example, a search coil.

Measuring and monitoring of magnetic fields are common techniques in other industries. The cancer treatment system 1 according to the present embodiments includes the search coil in order to measure a magnetic field that fluctuates at a frequency of 230 kHz and has a magnetic flux density of about 20 mT. The cancer treatment system 1 measures the alternating current voltage induced by the alternating current magnetic field in the search coil, and obtains the magnitude of the magnetic field, specifically, the magnetic flux density, using, for example, a correspondence table between voltage and magnetic field. In the cancer treatment system 1, the magnetic field may be measured by a gaussmeter.

The magnetic field measurer 107 is supported by a support unit 111. As illustrated in FIG. 6, the support unit 111 is movable in the respective directions of the X, Y, and Z axes in a three-dimensional coordinate system composed of mutually orthogonal X, Y, and Z axes. Specifically, the support unit 111 moves the magnetic field measurer 107 by a set distance along the X-axis direction Dx, the Y-axis direction Dy, and the Z-axis direction Dz.

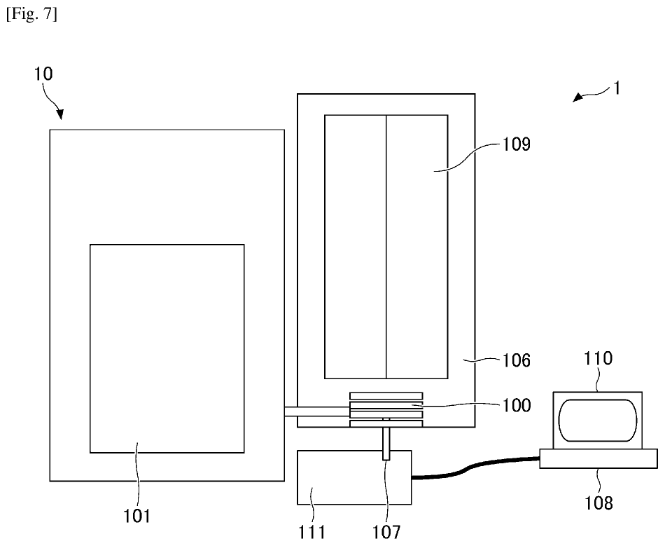

Magnetic field measurement using the magnetic field measurer 107 in the cancer treatment system 1 will be described. First, the magnetic field measurement performed before treatment in the cancer treatment system 1 will be described. FIG. 7 is a top view illustrating the magnetic field measurement performed before treatment in the cancer treatment system 1 according to the first embodiment.

In the cancer treatment system 1, a magnetic field generated by a magnetic field generator 100 is measured before a patient P is treated.

On the other hand, the magnetic field generator 100 generates a magnetic field by the coil 100a. The magnetic flux interlinked with a coil such as the coil 100a changes even a slight difference in position. Therefore, it is necessary that the magnetic field at the position where the affected area is actually positioned in the magnetic field generator 100 by taking actual measurements.

In the cancer treatment system 1 according to the first embodiment, before the patient P is treated, the magnetic field at the position where the affected area of the patient P is positioned in the magnetic field generator 100 is measured using the magnetic field measurer 107.

The cancer treatment system 1 includes a detection circuit (not illustrated) that detects a voltage generated by a magnetic field in a search coil that is the magnetic field measurer 107, a magnetic field intensity recorder 108 that acquires a voltage signal obtained by the detection circuit and then calculates and records the magnetic field intensity, and a magnetic field intensity display 110 that displays the magnitude of the magnetic field recorded by the magnetic field intensity recorder 108.

The cancer treatment system 1 controls the support unit 111 to measure the magnetic field generated in the magnetic field generator 100 by the magnetic field measurer 107. Then, the cancer treatment system 1 records the measurement result by the magnetic intensity recorder 108.

The magnetic field in the vicinity of the magnetic field generator 100 is also measured in order to estimate the magnetic field at the position (treatment position) where the affected area of the patient P is positioned during treatment of the patient P to be described later. Then, the correspondence relationship between the magnetic field at the position where the affected part of the patient P is positioned and the magnetic field in the vicinity of the magnetic field generator 100 is calculated.

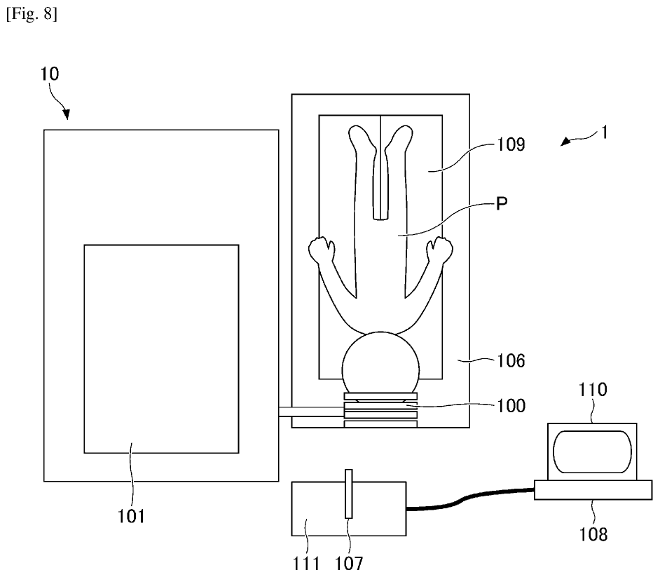

Next, magnetic field measurement performed while treatment in the cancer treatment system 1 will be described. FIG. 8 is a top view illustrating magnetic field measurement performed during treatment in the cancer treatment system 1 according to the first embodiment.

As described above, in the cancer treatment system 1, the amount of the magnetic field applied to the affected area is very important in relation to the treatment effect. Therefore, it is necessary to confirm by measurement whether a magnetic field having the desired magnetic flux density is applied to the affected area during actual treatment in the cancer treatment system 1.

In the cancer treatment system 1 according to the first embodiment, the magnetic field in the vicinity of the magnetic field generator 100 is measured using the magnetic field measurer 107 during treatment of the patient P. In the cancer treatment system 1, the magnetic field measurer 107 is disposed in the vicinity of the magnetic field generator 100, that is, around the treatment position. The cancer treatment system 1 estimates the magnetic field at the position where the affected area of patient P is positioned in magnetic field generator 100 by measuring the magnetic field in the vicinity of magnetic field generator 100.

During the actual treatment of patient P, the magnetic field itself at the position where the affected area of patient P is positioned cannot be measured because the affected area of patient P is in the magnetic field generator 100. The cancer treatment system 1 estimates the magnetic field at the position where the affected area of patient P is positioned based on the measurement result of the magnetic field in the vicinity of the magnetic field generator 100.

The magnetic field intensity recorder 108 is, for example, a personal computer. The magnetic field intensity recorder 108 converts a voltage signal generated in the search coil by the magnetic field into a magnetic flux density by a software program. For example, the magnetic field intensity recorder 108 may convert the voltage signal into the magnetic flux density using a correspondence table, or may convert the voltage signal into the magnetic flux density using a conversion formula.

In addition, the magnetic field intensity recorder 108 may determine whether the magnetic flux density is in a range from a predetermined upper limit value to predetermined lower limit value of a target magnetic flux density. For example, the magnetic field intensity recorder 108 may generate a warning sound or may display a warning on the magnetic field intensity display 110 if the magnetic field intensity exceeds the predetermined upper limit value or falls below the predetermined lower limit value of the target.

The magnetic field intensity recorder 108 can warn and make a healthcare worker such as a medical doctor who is performing the treatment aware of the warning. The healthcare worker can make reach a decision, based on the warning, to discontinue the treatment. In addition, in cancer treatment system 1, the current value output by power supply 101 may be adjusted such that the magnetic field intensity falls within the range between the upper limit value and the lower limit value. The adjustment of the current output by the power supply 101 may be automatically performed from the magnetic field intensity recorder 108, or may be manually set by the healthcare worker who performs the treatment.

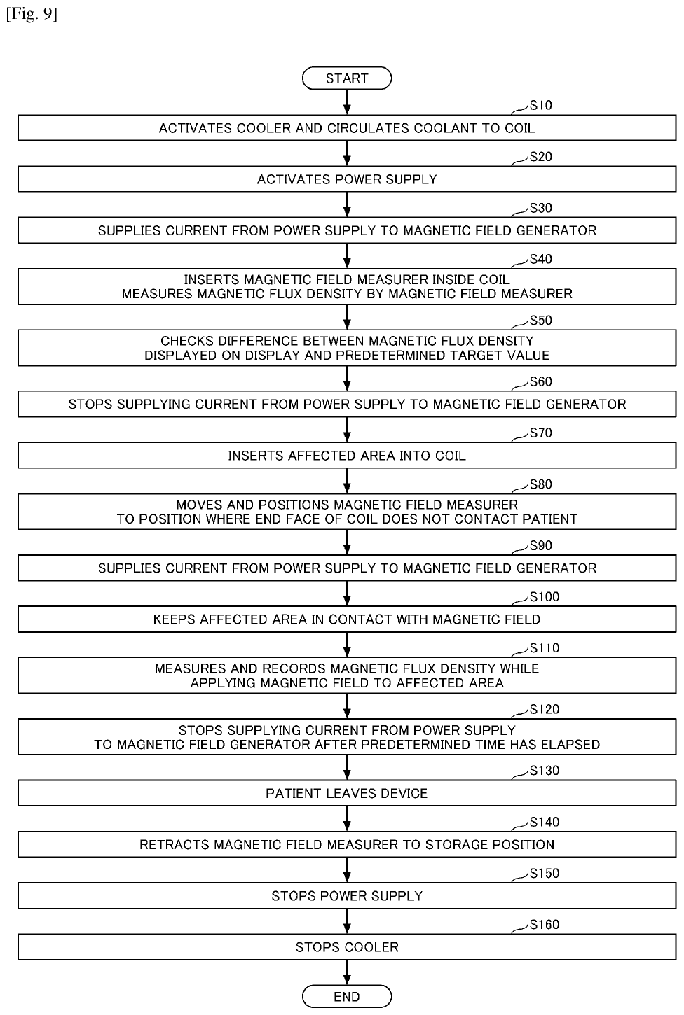

Next, a process of the cancer treatment system 1 will be described. FIG. 9 is a flowchart for explaining magnetic field measurement processing in the cancer treatment system 1 according to the first embodiment.

### Step S10

First, the cooler 103 is activated. When the power supply of cooler 103 is turned on to start the cooler 103, the cooler 103 adjusts the temperature of coolant to a predetermined target temperature. The cooler 103 supplies the temperature-adjusted coolant to the coil 100a of the magnetic field generator 100. The cooler 103 recovers the coolant supplied to the coil 100a. That is, the coolant whose temperature is adjusted by the cooler 103 circulates between the cooler 103 and the coil 100a of the magnetic field generator 100.

### Step S20

Next, the power supply 101 is activated. The power supply 101 is turned on so that power can be supplied from the power supply 101 to the magnetic field generator 100.

### Step S30

Next, the power supply 101 starts supplying a current to the coil 100a included in the magnetic field generator 100. For example, a current supply switch included in the power supply 101 is turned on. When the current supply switch is turned on, the power supply 101 starts supplying a current to the coil 100a.

### Step S40

Next, the support unit 111 is controlled to insert the magnetic field measurer 107 into the coil 100a of the magnetic field generator 100. Then, the magnetic field measurer 107 measures the magnetic flux density inside the coil 100a, that is, at the position where the affected area of patient P is positioned during treatment. For example, when the magnetic field measurer 107 is a search coil, an alternating current voltage generated in the search coil is measured.

The magnetic field measurer 107 may be inserted at a plurality of positions inside the coil 100a or at a point on the central axis of the coil 100a.

The magnetic field intensity recorder 108 records the result measured by the magnetic field measurer 107. The magnetic field intensity recorder 108 displays the measurement result on the magnetic field intensity display 110.

The support unit 111 inserts the magnetic field measurer 107 into the coil 100a of the magnetic field generator 100. The support unit 111 may be inserted by automatically extending an arm or the like by a motor, for example. Alternatively, a person who measures the magnetic field may directly insert the magnetic field measurer 107 into the coil 100a.

### Step S50

Next, the person who measures the magnetic field confirms a difference between the measurement result of the magnetic flux density displayed on the magnetic field intensity display 110 and the predetermined target value of the magnetic flux density. When the difference between the measured value of the magnetic flux density and the target value of the magnetic flux density is within the prescribed range and problem is not found on the measurement, the magnetic field measurer 107 is retracted from the inside of the coil 100a. When the difference between the measured value of the magnetic flux density and the target value of the magnetic flux density is out of the prescribed range, that is, when the measured value is out of the predetermined upper limit value or predetermined lower limit value, for example, the predetermined current value of the power supply 101 is adjusted.

The predetermined current value of the power supply 101 may be automatically adjusted by connecting the magnetic field intensity recorder 108 and the power supply 101, for example.

### Step S60

Next, the current supplied from the power supply 101 to the magnetic field generator 100 is stopped. For example, a current supply switch included in the power supply 101 is turned off. When the current supply switch is turned off, the power supply 101 stops supplying the current to the coil 100a.

Note that the steps S10, S20, S30, S40, S50, and S60 are pre-treatment processing.

### Step S70

Next, the patient P lies supine on the treatment table 106 of the cancer treatment device 10. Here, it is assumed that the patient P has an affected area on the head. For example, it is assumed that the affected area of patient P is brain cancer. The patient P inserts his or her head, which is an affected part, into the coil 100a of the magnetic field generator 100.

### Step S80

Next, the support unit 111 moves the magnetic field measurer 107 to the vicinity of the magnetic field generator 100 and determines the position. The magnetic field measurer 107 is positioned in the vicinity of the end surface of the coil 100a and at a position not in contact with the patient P. The magnetic field measurer 107 is positioned at an appropriate position depending on, for example, the position of the affected area of patient P.

During the treatment, the support unit 111 moves and fixes the magnetic field measurer 107 to an appropriate position outside the coil 100a so as not to contact the patient.

### Step S90

Next, the power supply 101 starts supplying a current to the coil 100a included in the magnetic field generator 100. That is, in the cancer treatment system 1, the power supply 101 supplies a current to the magnetic field generator 100. For example, a current supply switch included in the power supply 101 is turned on. When the current supply switch is turned on, the power supply 101 starts supplying a current to the coil 100a.

### Step S100

The magnetic field generator 100 continuously applies a magnetic field to the affected area of patient P for a certain period of time. In other words, the magnetic field generator 100 maintains a state in which the magnetic field is applied to the affected area of patient P for a certain period of time. For example, when the treatment time is 30 minutes, the magnetic field generator 100 applies the magnetic field to the affected area of patient P for 30 minutes. While the magnetic field generator 100 is applying a magnetic field, the patient P should keep still as much as possible.

### Step S110

The cancer treatment system 1 records the magnetic flux density measured by the magnetic field measurer 107 while applying the magnetic field to the affected area of patient P. The magnetic field measurer 107 measures the magnetic flux density while applying the magnetic field to the affected area of patient P, and outputs the measurement result to the magnetic field intensity recorder 108. The magnetic field intensity recorder 108 records the result measured by the magnetic field measurer 107. For example, when the magnetic field measurer 107 is a search coil, the magnetic field intensity recorder 108 acquires an output voltage measured by the search coil. Then, the magnetic field intensity recorder 108 calculates the magnetic flux density at the position where the search coil is positioned by a correspondence table or a formula. Then, from the calculated magnetic flux densities, the magnetic flux density at the position where the affected area of patient P is positioned in the coil 100a is calculated by the correspondence table or the formula.

During the treatment, it is necessary to monitor that the same magnetic flux density as the magnetic flux density measured in step S40 is generated at the center of the coil from the calculated magnetic flux density. The magnetic field intensity recorder 108 records the value of the magnetic flux density at an appropriate time interval, for example, every minute.

### Step S120

After a predetermined period of time has elapsed and the magnetic field is continuously applied to the affected area of patient P for the predetermined period of time, the current supplied from power supply 101 to magnetic field generator 100 is stopped and the treatment is ended. For example, if the predetermined treatment time is 30 minutes, the treatment is ended when the magnetic field generator 100 applies the magnetic field for 30 minutes.

### Step S130

The patient P then sits up and gets off the treatment table 106. Then, the patient P leaves the cancer treatment device 10. The patient P away from the cancer treatment device 10 moves to, for example, a waiting room or the like.

### Step S140

The magnetic field measurer 107 is retracted to a predetermined storage position. Then, the magnetic field measurer 107 stops the operation.

### Step S150

Next, the power supply 101 stops the operation. Specifically, the power of the power supply 101 is stopped. For example, a power switch included in the power supply 101 is turned off.

### Step S160

Next, the cooler 103 stops its operation. Specifically, the power of the cooler 103 is stopped. For example, a power switch included in the cooler 103 is turned off.

The magnetic field intensity recorder 108 may compare the magnetic field intensity measured during the treatment with the predetermined upper limit value and the predetermined the lower limit value and determine whether the magnetic field intensity is normal or abnormal by comparing the results. If a situation, in which the magnetic flux density being monitored exceeds the predetermined upper limit value or falls below the predetermined lower limit value, occurs during treatment, the cancer treatment system 1 may immediately notify a healthcare worker such as a medical doctor who performs treatment by a warning sound or by displaying a warning on the magnetic field intensity display 110. The healthcare worker can make reach a decision, based on the warning, to discontinue the treatment.

The treatment by the cancer treatment device 10 is performed, for example, three times a week for three weeks with the above-described treatment counted as one time. Then, the effect of suppressing proliferation of cancer cells or suppressing cancers by the treatment is confirmed by Magnetic Resonance Imaging (MRI), simple X-ray Computer Tomography (simple X-ray CT), contrast computed tomography, Positron Emission Tomography (PET), or the like. The treatment is repeated as necessary. For this purpose, how much and for how many hours the magnetic flux density is cumulatively applied to the affected area is recorded as a treatment result by monitoring during the treatment.

### Summary

According to the cancer treatment system 1 of the present embodiments, in the cancer treatment device 10 that suppresses proliferation of cancer cells by an alternating current magnetic field, it is possible to record whether or not a magnetic flux density having a magnitude appropriate for a treatment purpose is applied to an affected area of a patient. In addition, according to the cancer treatment system 1, it is possible to record treatment results in the cancer treatment device 10. Furthermore, according to the cancer treatment system 1, it is possible to provide a doctor with information on the treatment results performed by the cancer treatment device 10.

The technique disclosed in Patent Document 1 is a cancer treatment device that applies a magnetic field to an affected area of a patient. Since the applied magnetic field is not visible, it is not visually known whether the actually required magnetic field is applied to the affected area. Therefore, it is necessary to monitor the magnitude of the magnetic field to determine whether appropriate treatment is being performed.

The cancer treatment system 1 includes a sensor for monitoring a treatment effect, specifically, a magnetic field measurer 107. The magnetic field measurer 107 includes, for example, a search coil or a gaussmeter. In the cancer treatment system 1, by measuring at least one of the magnetic flux density at the position of the affected area before the treatment and the magnetic flux density at a position where the affected area does not contact the patient during the treatment, it is possible to record whether or not the magnetic flux density having a magnitude suitable for the purpose of the treatment is applied to the affected area.

The cancer treatment system 1 can be applied as a human cancer treatment device. The cancer treatment system 1 is particularly applicable to the treatment of cancer types such as malignant glioblastoma (glioblastoma) for which the current surgical and chemotherapeutic treatments are less effective. In addition, a treatment target to be treated by the cancer treatment system 1 is not limited to a person, but may be a pet such as a dog or a cat, or a domestic animal such as a horse. In addition, the type of cancer targeted by the cancer treatment system 1 is also applicable to cancers other than glioblastoma. For example, the cancer treatment system 1 may be applied to any treatment of glioblastomas, gliomas, pancreatic cancer, breast cancers, malignant melanomas, malignant mesothelioma, and oral cancers.

On the other hand, as a known medical device, a hyperthermia device for treating cancers by applying a magnetic field to generate heat has been known. However, hyperthermia monitors the temperature but not the magnitude of the magnetic field because the effect of treatment is determined by the increase in temperature of the affected area.

By measuring the magnitude of the magnetic field, the cancer treatment system 1 can monitor whether the originally required magnitude of the magnetic field is applied to the cancer affected area before and during the treatment. In addition, when an abnormality occurs in which the magnitude of the magnetic field being monitored exceeds the predetermined upper limit value or falls below the predetermined lower limit value, the cancer treatment system 1 can issue warning information to a healthcare worker such as a medical doctor to discontinue the treatment. Furthermore, according to the cancer treatment system 1, the magnitude of the monitored magnetic field is recorded as a treatment result, and it is also possible to provide a treatment record indicating how much and for how many hours the magnitude of the magnetic field is applied to the affected area in a cumulative manner with respect to continuous treatment.

In the cancer treatment system 1, for example, the magnetic field measurer 107 may use a search coil in the magnetic field measurement before treatment, and the magnetic field measurer 107 may use a gaussmeter in the magnetic field measurement during treatment.

The magnetic field generator 100 generates a magnetic field having a magnetic flux density of several mT. It is desirable to use a search coil to measure a magnetic field having a strong magnetic flux density of about several mT. Therefore, when the magnetic flux density inside the coil 100a is measured before treatment, it is desirable to use the search coil.

On the other hand, in the magnetic field measurement during the treatment, the measurement is performed at a position away from the coil 100a by, for example, about 15 centimeters so as not to contact the patient P. Since the magnetic flux density becomes 1 mT or less away from the coil 100a, a commercially available gaussmeter may be used to measure the magnetic field at a position where the magnetic flux density is low. For example, a gaussmeter may be fixed at a position about 15 centimeters away from the end of the coil 100a so that the magnitude of the magnetic field can be measured at all times before and during treatment.

A Hall element, a magnetoresistance effect element, or the like may be used as the magnetic field measurer 107.

### Cancer Treatment System 2

In the cancer treatment device 10, when an alternating current magnetic field having a frequency of 230 kHz and a magnetic flux density of about 20 mT is applied to an affected area, in a medium-sized animal such as a human or a dog, the liquid in the tissue generates heat little by little and the body temperature rises. The inventors of the present invention have found that, in cancer treatment device 10, the larger the cross-sectional area of the living body in which the magnetic flux is interlinked, the more easily heat is generated. In the cancer treatment device 10, the larger the cross-sectional area of the living body where the magnetic flux is interlinked, the easier it is to generate heat, because the water that makes up about 70% of the living body is an electrolytic solution with electrical conductivity, which generates eddy currents due to electromagnetic induction.

Therefore, in the cancer treatment device 10, it is necessary to constantly monitor the state of heat generation due to the eddy current while the alternating current magnetic field is applied to the affected area. In particular, in the cancer treatment device 10, the temperature needs to be constantly monitored. In the cancer treatment device 10, when the temperature of the portion to which the alternating current magnetic field is applied exceeds the predetermined upper limit value, it is necessary to stop the treatment or temporarily stop the generation of the alternating current magnetic field. Therefore, the cancer treatment system 2 according to the second embodiment includes a temperature measurer 207 that measures the temperature of the affected area or around the affected area to which the magnetic field is applied by the magnetic field generator 100.

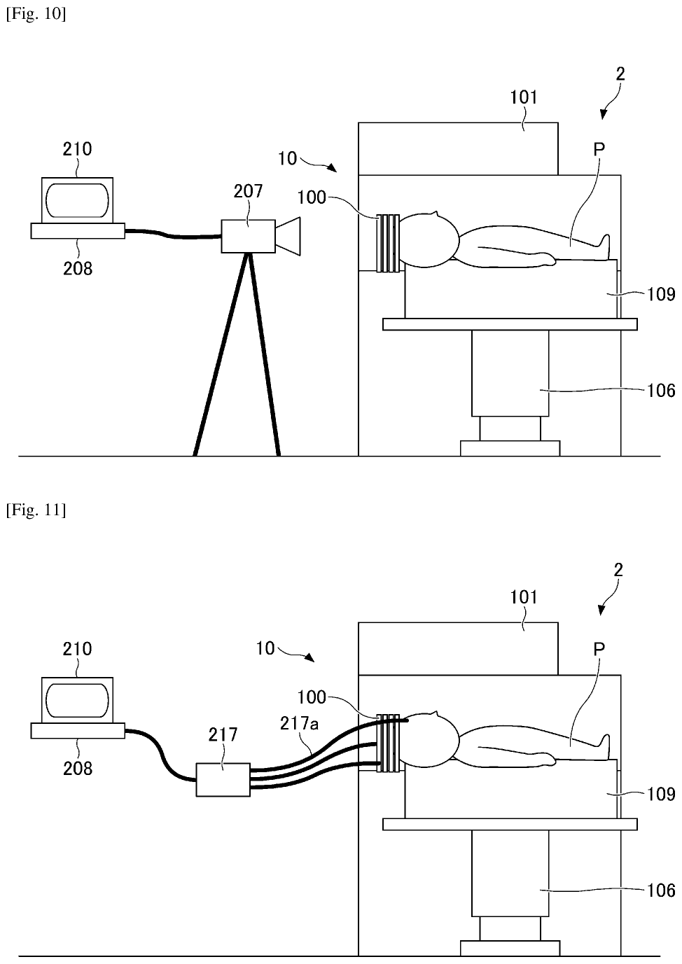

The cancer treatment system 2 includes the cancer treatment device 10 and at least one of temperature measurer 207 and temperature measurer 217 for measuring the body temperature of patient P. FIG. 10 is a side view illustrating a case where a thermoviewer is used for temperature measurement in the cancer treatment system 2 according to the second embodiment. The temperature measurer 207 is, for example, a thermoviewer. The temperature measurer 207 is connected to the temperature recorder 208. The temperature measurer 207 outputs the measured data to the temperature recorder 208. The temperature recorder 208 acquires temperature information measured by the thermoviewer which is the temperature measurer 207. The temperature display 210 displays the temperature distribution acquired by the temperature recorder 208.

The temperature measurer 207, which is a thermoviewer, measures the surface temperature of the affected area or around the affected area from a position at least 15 centimeters away from the magnetic field generator 100.

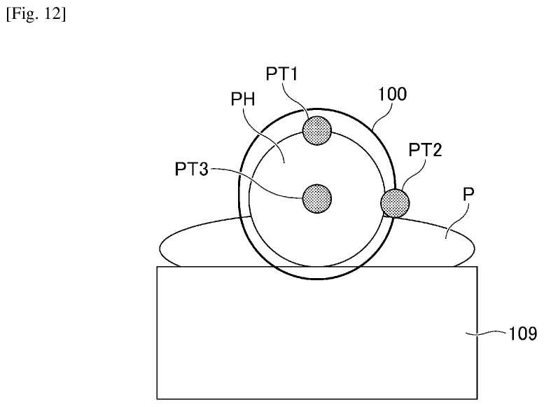

The temperature measurer 207 is not limited to a thermoviewer. FIG. 11 is a side view illustrating a case where an optical fiber thermometer is used for temperature measurement in the cancer treatment system 2 according to the second embodiment. The temperature measurer 217 is an optical fiber thermometer. The tip of the optical fiber 217a of the temperature measurer 217 is brought into contact with and fixed to a plurality of positions such as an affected part, the vicinity of the affected part, and a magnetic field generation device. The temperature measurer 217 outputs the measured data to the temperature recorder 208.

FIG. 12 is a front view illustrating an installation location of an optical fiber 217a included in the temperature measurer 217, which is an optical fiber thermometer, for temperature measurement in the cancer treatment system 2 according to the second embodiment.

The tip of the optical fibers 217a included in the temperature measurer 217, which is an optical fiber thermometer, are located at, for example, each of the forehead PT1, the temporal region PT2, and the parietal region PT3 of the head PH of the patient P. Regarding the tip of the temperature measurer 217, which is an optical fiber thermometer, the above example is merely an example. For example, the tip of the optical fiber 217a of the temperature measurer 217, which is an optical fiber thermometer, may be fixed to at least one of the forehead PT1, the temporal region PT2, and the parietal region PT3. In addition, for example, the tip of the optical fiber 217a of the temperature measurer 217, which is an optical fiber thermometer, may be fixed to a place other than the forehead PT1, the temporal region PT2, and the parietal region PT3.

The temperature recorder 208 acquires temperature information measured by the temperature measurer 217. The temperature display 210 constantly displays the temperature information acquired by the temperature recorder 208 during the treatment.

The temperature recorder 208 determines whether the measured temperature information is in between a predetermined upper limit value and a predetermined lower limit value of a target temperature by a software program. The temperature recorder 208 may determine whether the measured temperature information is normal or abnormal by determining whether the measured temperature information is in a range from a predetermined upper limit value to predetermined lower limit value of a target temperature.

If the temperature exceeds the predetermined upper limit value or falls below the predetermined lower limit value of the target, the temperature recorder 208 may generate a warning sound or may display a warning on the temperature display. When the temperature recorder 208 issues a warning, the temperature recorder 208 can make a healthcare worker such as a medical doctor who is performing the treatment aware the notification. When the temperature recorder 208 issues the warning, the healthcare worker can select, for example, to discontinue the treatment. In addition, the current value output by power supply 101 may be adjusted such that the temperature falls within the range between the upper limit value and lower limit value. The adjustment of the current value of the power supply 101 may be automatically adjusted, or may be manually set by the healthcare worker who performs the treatment.

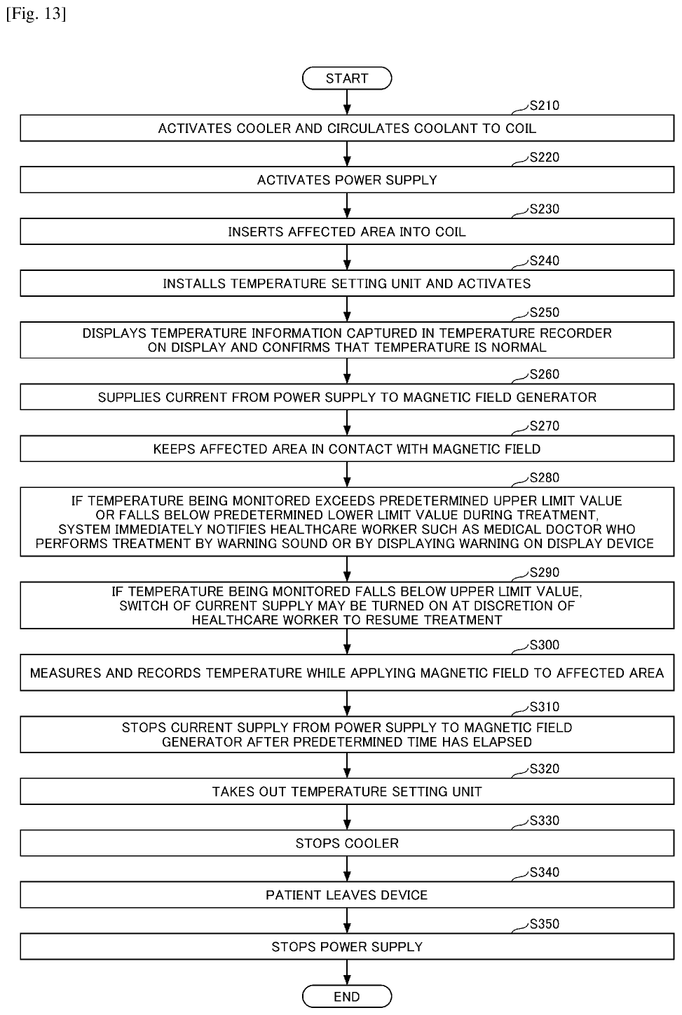

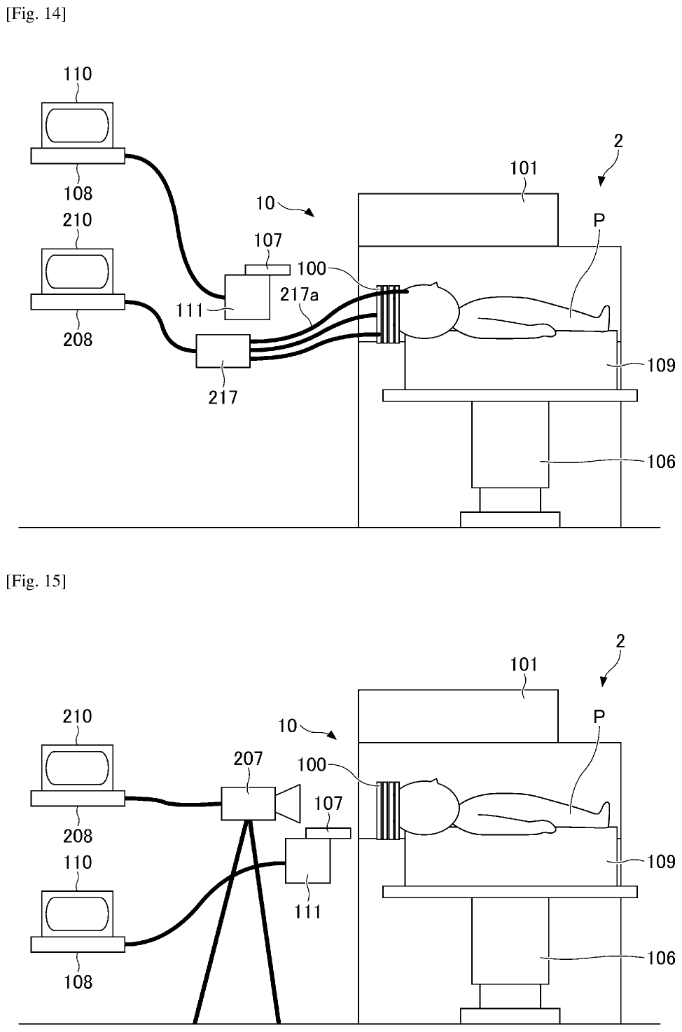

Next, a process of the cancer treatment system 2 will be described. FIG. 13 is a flowchart for explaining temperature measurement processing in the cancer treatment system 2 according to the second embodiment.

### Step S210

First, the cooler 103 is activated. When the power supply of cooler 103 is turned on to start the cooler 103, the cooler 103 adjusts the temperature of coolant to a predetermined target temperature. The cooler 103 supplies the temperature-adjusted coolant to the coil 100a of the magnetic field generator 100. The cooler 103 recovers the coolant supplied to the coil 100a. That is, the coolant whose temperature is adjusted by the cooler 103 circulates between the cooler 103 and the coil 100a of the magnetic field generator 100.

### Step S220

Next, the power supply 101 is activated. The power supply 101 is turned on so that power can be supplied from the power supply 101 to the magnetic field generator 100.

### Step S230

Next, the patient P lies supine on the treatment table 106 of the cancer treatment device 10. Here, it is assumed that the patient P has an affected area on the head. For example, it is assumed that the affected area of patient P is a brain cancer. The patient P inserts his or her head, which is an affected part, into the coil 100a of the magnetic field generator 100.

### Step S240

Next, a temperature measurer is installed. First, a case where the temperature measurer 207 is used will be described. The temperature measurer 207 is installed at a position where the temperature of the affected area can be measured. Further, the position of the installed temperature measurer 207 is fixed. Then, the temperature measurer 207 is activated.

Next, a case where the temperature measurer 217 is used will be described. The tip of the optical fiber 217a of the optical fiber thermometer which is the temperature measurer 217 is fixed to the affected part, around the affected area, the wall surface of the magnetic field generator 100, and the like. Then, the temperature measurer 217 is activated.

### Step S250

Next, the cancer treatment system 2 displays the temperature information captured in the temperature recorder 208 on the temperature display 210 to confirm that the surface temperature of the affected area is at normal values before treatment begins.

The steps S210, S220, S230, S240, and S250 are pre-treatment processing.

### Step S260

Next, the power supply 101 starts supplying a current to the coil 100a included in the magnetic field generator 100. For example, a current supply switch included in the power supply 101 is turned on. When the current supply switch is turned on, the power supply 101 starts supplying a current to the coil 100a.

### Step S270

The magnetic field generator 100 continuously applies a magnetic field to the affected area of patient P for a certain period of time. In other words, the magnetic field generator 100 maintains a state in which the magnetic field is applied to the affected area of patient P for a certain period of time. For example, when the treatment time is 30 minutes, the magnetic field generator 100 applies the magnetic field to the affected area of patient P for 30 minutes. While the magnetic field generator 100 is applying a magnetic field, the patient P should keep still as much as possible.

### Step S280

If the temperature being monitored exceeds the predetermined upper limit value or falls below the predetermined lower limit value during the treatment, the temperature recorder 208 immediately notifies a healthcare worker such as a medical doctor who performs the treatment by a warning sound or by displaying a warning on the temperature display 210. This allows the healthcare worker to discontinue the treatment. The healthcare worker may also switch off the current supply once.

### Step S290

In the temperature recorder 208, if the temperature being monitored falls below the upper limit value, the switch of the current supply may be turned on at the discretion of the healthcare worker to resume the treatment.

### Step S300

The healthcare worker monitors the temperature information displayed on the temperature display 210. The temperature recorder 208 records the temperature information at appropriate time intervals, for example, every minute.

### Step S310

After a predetermined time has elapsed and the magnetic field is continuously applied to the affected area of patient P for the predetermined period of time, the current supplied from power supply 101 to magnetic field generator 100 is stopped and the treatment is ended. For example, if the predetermined treatment time is 30 minutes, the treatment is ended when the magnetic field generator 100 applies the magnetic field for 30 minutes.

### Step S320

Next, the healthcare worker removes the temperature measurer. The tip of the optical fiber 217a included in the temperature measurer 217, which is an optical fiber thermometer, is removed. Further, the healthcare worker moves the temperature measurer 207, which is a thermoviewer, to a position where the patient can easily move.

### Step S330

Next, the cooler 103 stops its operation. Specifically, the power supply of the cooler 103 is stopped. For example, a power switch included in the cooler 103 is turned off.

### Step S340

The patient P then sits up and gets off the treatment table 106. Then, the patient P leaves the cancer treatment device 10. The patient P away from the cancer treatment device 10 moves to, for example, a waiting room or the like.

### Step S350

Next, the power supply 101 stops its operation. Specifically, the power supply of the power supply 101 is stopped. For example, a power switch included in the power supply 101 is turned off.

### Summary

According to the cancer treatment system 2 of the present embodiments, it is possible to constantly monitor the state of heat generation due to eddy current in the cancer treatment device 10. In particular, the cancer treatment system 2 can constantly monitor the temperature of a portion including the affected area to which the alternating current magnetic field is applied. In addition, according to the cancer treatment system 2 of the present embodiments, in the cancer treatment device 10, when the temperature of the portion to which the alternating current magnetic field is applied exceeds the predetermined upper limit value, the treatment can be stopped or the generation of the alternating current magnetic field can be temporarily stopped.

In the above description, the cancer treatment system 2 includes both the temperature measurer 207 and the temperature measurer 217, but may include any one of the temperature measurer 207 and the temperature measurer 217.

In the above description, when the temperature is equal to or higher than the upper limit value, the cancer treatment system 2 stops the treatment or temporarily stops the generation of the alternating current magnetic field. However, the affected area may be cooled without stopping the treatment or temporarily stopping the generation of the alternating current magnetic field. For example, when the temperature is equal to or higher than the upper limit value, the cancer treatment system 2 may cool the affected area by supplying cooled air to the affected area, or may cool the affected area by supplying cooling water to a cooling bag that cools the affected area.

### Modification

In the above description, the cancer treatment system 1 including the magnetic field measurer 107 and the cancer treatment system 2 including the temperature measurer 207 and the temperature measurer 217 have been described. However, the cancer treatment system may perform both magnetic field measurement and temperature measurement.

In other words, a cancer treatment system includes: a magnetic field generator 100 configured to generate an alternating current magnetic field to be applied to a cancer affected area; a magnetic field measurer 107 configured to measure a magnitude of the magnetic field generated by the magnetic field generator 100; and a temperature measurer 207 or 217 configured to measure a temperature around the cancer affected area including the cancer affected area, wherein the cancer affected area is treated by inserting the cancer affected area into the magnetic field generated by the magnetic field generator 100 without using a heat-generating medium and without depending on a hyperthermic effect using a heat-generating action of the magnetic field.

The temperature measurer 207 or 217 is usually used for the purpose of measuring temperature changes during an application of magnetic field (during treatment operation). On the other hand, the magnetic field measurer 107 is mainly used for adjustment or operation check prior to treatment, and the magnetic field measurer 107 can also be used during the treatment. When the magnetic field measurer 107 is used during the treatment, the magnitude of magnetic field intensity of the affected area of a patient P is needed to be assumed from a magnetic field intensity near the affected area of patient P because the position where the magnetic field intensity is needed to be measure is occupied by the affected area of patient P.

The frequency of the alternating current magnetic field and the magnitude of the magnetic field in the cancer treatment device 10 are not limited to the values described above, and treatment may be performed at other frequencies and other magnitudes of the magnetic field.

In addition, although an example in which the patient P is treated in a supine position has been described above, the patient P may be treated in a seated state. When the patient P receives treatment in a seated state, the coil 100a of the magnetic field generator 100 is fixed in a state rotated by 90° with respect to the vertical direction in FIG. 1, that is, the coil 100a of the magnetic field generator 100 is positioned above the head of the patient P. It is equally feasible to measure and monitor the magnitude of the magnetic field when the patient P is undergoing treatment in a seated position.

The magnetic field measurement may be performed at the time of maintenance and inspection of the cancer treatment device. Furthermore, whether the magnetic field measurement is performed in the procedure described above depends on an instruction from the Ministry of Health, Labour and Welfare at the time of approval of the cancer treatment device.

Embodiments of the present disclosure include the following aspects.

<1> A cancer treatment system includes a magnetic field generator configured to generate an alternating current magnetic field to be applied to a cancer affected area, and a magnetic field measurer configured to measure a magnitude of the magnetic field generated by the magnetic field generator, wherein the cancer affected area is inserted into the magnetic field generated by the magnetic field generator to treat cancers.

<2> A cancer treatment system includes a magnetic field generator configured to generate an alternating current magnetic field to be applied to a cancer affected area, and a temperature measurer configured to measure a temperature of the cancer affected area or around the cancer affected area, wherein the cancer affected area is inserted into the magnetic field generated by the magnetic field generator to treat cancers without using a heat-generating medium and without depending on hyperthermic effect using a heat-generating action by the magnetic field.

<3> The cancer treatment system according to <1>, further includes a magnetic field intensity recorder configured to record a magnetic field intensity measured by the magnetic field measurer.

<4> The cancer treatment system according to <2>, further includes a temperature recorder configured to record a temperature measured by the temperature measurer.

<5> The cancer treatment system according to <3>, wherein the magnetic field intensity recorder determines whether the magnetic field intensity measured is normal or abnormal based on a result of comparing a predetermined upper limit value of a target magnetic field intensity and a predetermined lower limit value of the target magnetic field intensity with the magnetic field intensity measured.

<6> The cancer treatment system according to <4>, wherein the temperature recorder determines whether the temperature measured is normal or abnormal based on a result of comparing a predetermined upper limit value of a target temperature and a predetermined lower limit value of the target temperature with the temperature measured.

<7> The cancer treatment system according to <1>, wherein the magnetic field measurer includes a search coil, and wherein the search coil is inserted to a position where magnetic field to be applied to treat cancers to measure the alternating current magnetic field before the magnetic field is applied to the cancer affected area.

<8> The cancer treatment system according to <1>, wherein the magnetic field measurer includes a search coil, and wherein the search coil is disposed around a position where magnetic field to be applied to treat cancers to measure the alternating current magnetic field while the magnetic field is applied to the cancer affected area.

<9> The cancer treatment system according to <1>, wherein the magnetic field measurer includes a search coil, wherein the search coil is inserted to a position where magnetic field to be applied to treat cancers to measure the alternating current magnetic field before the magnetic field is applied to the cancer affected area, and wherein the search coil is disposed around the position where magnetic field to be applied to treat cancers to measure the alternating current magnetic field while the magnetic field is applied to the cancer affected area.

<10> The cancer treatment system according to <5> or <6>, wherein the magnetic field generator includes a coil configured to generate the alternating current magnetic field, wherein the cancer treatment system further comprises a power supply configured to supply an alternating current voltage to the coil, and wherein when the result is abnormal, a current supplied from the power supply to the magnetic field generator is stopped.

<11> The cancer treatment system according to <5> or <6>, wherein the magnetic field generator includes a coil configured to generate the alternating current magnetic field, wherein the cancer treatment system further comprises a power supply configured to supply an alternating current voltage to the coil, and wherein when the result is abnormal, a magnitude of current supplied from the power supply to the magnetic field generator is adjusted.

<12> The cancer treatment system according to <1>, wherein the magnetic field measurer includes a gaussmeter.

<13> The cancer treatment system according to <1>, wherein the magnetic field measurer includes a Hall element.

<14> The cancer treatment system according to <1>, wherein the magnetic field measurer includes a magnetoresistance effect element.

<15> The cancer treatment system according to <4>, wherein the temperature measurer includes a thermoviewer, and wherein the thermoviewer measures a surface temperature of the cancer affected area and around the cancer affected area from a position at least 15 centimeters away from the magnetic field generator, and outputs measurement data to the temperature recorder.

<16> The cancer treatment system according to <4>, wherein the temperature measurer includes an optical fiber thermometer, and wherein the optical fiber thermometer measures a surface temperature of the cancer affected area and around the cancer affected area, and outputs measurement data to the temperature recorder.

<17> The cancer treatment system according to <3>, further includes a magnetic field intensity display configured to display a magnitude of the magnetic field recorded by the magnetic field intensity recorder.

<18> The cancer treatment system according to <2>, further includes a temperature display configured to display a temperature.

<19> The cancer treatment system according to any one of <1> to <18>, further includes a cooler configured to cool the magnetic field generator.

<20> The cancer treatment system according to <19>, wherein the magnetic field generator includes a hollow coil, and wherein the cooler supplies a coolant to the coil.

<21> The cancer treatment system according to any one of <1> to <20>, wherein the cancers include any of glioblastomas, gliomas, pancreatic cancer, breast cancers, malignant melanomas, malignant mesothelioma, and oral cancers.

<22> A cancer treatment system includes a magnetic field generator configured to generate an alternating current magnetic field to be applied to a cancer affected area, a magnetic field measurer configured to measure a magnitude of the magnetic field generated by the magnetic field generator, and a temperature measurer configured to measure a temperature around the cancer affected area including the cancer affected area, wherein the cancer affected area is treated by inserting the cancer affected area into the magnetic field generated by the magnetic field generator without using a heat-generating medium and without depending on a hyperthermic effect using a heat-generating action of the magnetic field.

This international application claims priority under Japanese Patent Applications No. 2022-075118, filed Apr. 28, 2022 and No. 2022-075130, filed Apr. 28, 2022, and the entire contents of Japanese Patent Applications No. 2022-075118 and No. 2022-075130 are incorporated herein by reference.

It should be noted that the present invention is not limited to the configurations described herein, such as combinations of the configurations described in the above embodiments and other elements. These points can be changed within a range not departing from the gist of the present invention, and can be appropriately determined according to the application form.

### Reference Signs List

1, 2 Cancer treatment system
10 Cancer treatment device
100 Magnetic field generator
100a Coil
100a1 Metallic pipe
100a2 Insulating sheet
100a3 Protection sheet
100b Piping
101 Power supply
102 Matching unit
103 Cooler
104 Manifold
105 Hose
106 Treatment table
107 Magnetic field measurer
108 Magnetic field intensity recorder
111 Support unit
109 Non-conductive buffer material
110 Magnetic field intensity display
208 Temperature recorder
210 Temperature display
207, 217 Temperature measurer
217a Optical fiber
P patient
PH Head
PT1 Forehead
PT2 Temporal region
PT3 Parietal region

### Citation List

### Patent Literature

[Patent Document 1] Japanese Patent No. 6603812.

[Patent Document 2] Japanese Patent Application Laid-Open No. 2019-201921.

1. A cancer treatment system comprising:
a magnetic field generator configured to generate an alternating current magnetic field to be applied to a cancer affected area; and
a magnetic field measurer configured to measure a magnitude of the magnetic field generated by the magnetic field generator,
wherein the cancer affected area is inserted into the magnetic field generated by the magnetic field generator to treat cancers.

2. A cancer treatment system comprising:
a magnetic field generator configured to generate an alternating current magnetic field to be applied to a cancer affected area; and
a temperature measurer configured to measure a temperature of the cancer affected area or around the cancer affected area;
wherein the cancer affected area is inserted into the magnetic field generated by the magnetic field generator to treat cancers without using a heat-generating medium and without depending on hyperthermic effect using a heat-generating action by the magnetic field.

3. The cancer treatment system according to claim 1, further comprising a magnetic field intensity recorder configured to record a magnetic field intensity measured by the magnetic field measurer.

4. The cancer treatment system according to claim 2, further comprising a temperature recorder configured to record a temperature measured by the temperature measurer.

5. The cancer treatment system according to claim 3,
wherein the magnetic field intensity recorder determines whether the magnetic field intensity measured is normal or abnormal based on a result of comparing a predetermined upper limit value of a target magnetic field intensity and a predetermined lower limit value of the target magnetic field intensity with the magnetic field intensity measured.

6. The cancer treatment system according to claim 4,
wherein the temperature recorder determines whether the temperature measured is normal or abnormal based on a result of comparing a predetermined upper limit value of a target temperature and a predetermined lower limit value of the target temperature with the temperature measured.

7. The cancer treatment system according to claim 1,
wherein the magnetic field measurer includes a search coil, and
wherein the search coil is inserted to a position where magnetic field to be applied to treat cancers to measure the alternating current magnetic field before the magnetic field is applied to the cancer affected area.

8. The cancer treatment system according to claim 1,
wherein the magnetic field measurer includes a search coil, and
wherein the search coil is disposed around a position where magnetic field to be applied to treat cancers to measure the alternating current magnetic field while the magnetic field is applied to the cancer affected area.

9. The cancer treatment system according to claim 1,
wherein the magnetic field measurer includes a search coil,
wherein the search coil is inserted to a position where magnetic field to be applied to treat cancers to measure the alternating current magnetic field before the magnetic field is applied to the cancer affected area, and
wherein the search coil is disposed around the position where magnetic field to be applied to treat cancers to measure the alternating current magnetic field while the magnetic field is applied to the cancer affected area.

10. The cancer treatment system according to claim 5,
wherein the magnetic field generator includes a coil configured to generate the alternating current magnetic field,
wherein the cancer treatment system further comprises a power supply configured to supply an alternating current voltage to the coil, and
wherein when the result is abnormal, a current supplied from the power supply to the magnetic field generator is stopped.

11. The cancer treatment system according to claim 5,
wherein the magnetic field generator includes a coil configured to generate the alternating current magnetic field,
wherein the cancer treatment system further comprises a power supply configured to supply an alternating current voltage to the coil, and
wherein when the result is abnormal, a magnitude of current supplied from the power supply to the magnetic field generator is adjusted.

12. The cancer treatment system according to claim 1,
wherein the magnetic field measurer includes a gaussmeter.

13. The cancer treatment system according to claim 1,
wherein the magnetic field measurer includes a Hall element.

14. The cancer treatment system according to claim 1,
wherein the magnetic field measurer includes a magnetoresistance effect element.

15. The cancer treatment system according to claim 4,
wherein the temperature measurer includes a thermoviewer, and
wherein the thermoviewer measures a surface temperature of the cancer affected area and around the cancer affected area from a position at least 15 centimeters away from the magnetic field generator, and outputs measurement data to the temperature recorder.

16. The cancer treatment system according to claim 4,
wherein the temperature measurer includes an optical fiber thermometer, and
wherein the optical fiber thermometer measures a surface temperature of the cancer affected area and around the cancer affected area, and outputs measurement data to the temperature recorder.

17. The cancer treatment system according to claim 3, further comprising a magnetic field intensity display configured to display a magnitude of the magnetic field recorded by the magnetic field intensity recorder.

18. (canceled)

19. The cancer treatment system according to claim 1, further comprising a cooler configured to cool the magnetic field generator.

20. (canceled)

21. The cancer treatment system according to claim 1,
wherein the cancers include any of glioblastomas, gliomas, pancreatic cancer, breast cancers, malignant melanomas, malignant mesothelioma, and oral cancers.

22. A cancer treatment system comprising:
a magnetic field generator configured to generate an alternating current magnetic field to be applied to a cancer affected area;
a magnetic field measurer configured to measure a magnitude of the magnetic field generated by the magnetic field generator; and
a temperature measurer configured to measure a temperature around the cancer affected area including the cancer affected area,
wherein the cancer affected area is treated by inserting the cancer affected area into the magnetic field generated by the magnetic field generator without using a heat-generating medium and without depending on a hyperthermic effect using a heat-generating action of the magnetic field.
